(19) United States
(12) Patent Application Publication
(10) Pub. No.: US 2025/0279211 A1
(43) Pub. Date: Sep. 4, 2025

(54) DIGITAL HEALTH ENGAGEMENT PLATFORM FOR WOMEN

(71) Applicant: Kimberly Smith, Irmo, SC (US)

(72) Inventor: Kimberly Smith, Irmo, SC (US)

(21) Appl. No.: 19/062,130

(22) Filed: Feb. 25, 2025

Related U.S. Application Data
(60) Provisional application No. 63/560,585, filed on Mar. 1, 2024.

(51) Int. Cl. G16H 50/30; G16H 10/20; G16H 10/60; G16H 40/67; G16H 80/00

(52) U.S. Cl. CPC G16H 50/30; G16H 10/20; G16H 10/60; G16H 40/67; G16H 80/00

(57) ABSTRACT

A system for improving the health and care coordination of women suffering from high blood pressure, diabetes and mental health conditions from pre-conception to menopause, the system including an app having instructions for accessing a digital health engagement platform. The platform has functionalities including health risk scoring, care plan management, care support, client reporting and interoperability. The system includes a Bluetooth enabled medical device such as a blood pressure monitor, for tracking and logging specific data that required to monitor overall health of a user wherein the Bluetooth enabled medical device is in communication with the computer readable medium. The system includes an index tool for measuring non-medical drivers of health, the index tool including a multi-question set that is pushed directly from a client portal to the application. The system integrates with an electronic health record system.

### CROSS-REFERENCE TO RELATED APPLICATIONS

This application is a Utility Patent application claiming priority to U.S. Provisional Patent Application Ser. No. 63/560,585, filed on Mar. 1, 2024, which is incorporated by reference herein in its entirety.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates to the field of software applications for the health of women from pre-conception to menopause, and more specifically to a platform for monitoring and coordinating the cardiovascular, diabetes, mental and non-medical drivers of health of the women. This digital health platform offers a health at home model for supporting women's health across the lifespan.

### 2) Description of related art

Globally, approximately 800 times a day, a woman or child dies from childbirth complications and 80% of these deaths happen after delivery.

Heart disease and stroke are the leading causes of maternal mortality. American Indian, Black, Hispanic, and Pacific Islander are more likely to experience adverse pregnancy outcomes, which exacerbates health disparities. In addition, black women are 2 to 3 times more likely to die from a pregnancy complication than white women.

Women who survive a complicated pregnancy are twice as likely to develop heart disease. According to a 2019 study by The Commonwealth Fund, the cost of maternal morbidity for all births in 2019 (from conception through age 5) is $32.3 billion and 75% of these costs stem from child outcomes. Healthy births have a vital impact on the long-term health and balanced utilization of healthcare for mom and baby. Research states “While maternal deaths in the United States number about 650 to 750 annually, severe maternal morbidity affects approximately 50,000 to 60,000 women each year, and the numbers are increasing.” For the 3.7 million births that occurred in 2021, as many as 480,000 women or more than 1 in 8 experienced maternal morbidity. This causes adverse maternal and child health outcomes and costs the United States over $32 billion from conception through age 5 for all children born in a single calendar year.

Early intervention and engagement (from preconception to menopause) can have a positive impact on lowering the risk of hypertension, postpartum depression/anxiety, and type 2 diabetes. Even-so, the art of addressing this problem is the acknowledgement that only 10 to 20% of health disparities, in general, are associated with direct medical care. Hence, 80% relates to non-medical care often attributed to social determinants of health. The cycle of poor outcomes is more often attributed to the lack of connectivity between what happens in the care environment as well as the consumer's personal environment.

The American College of Obstetricians and Gynecologists recommends that all women have contact with their obstetrician-gynecologists or other obstetric care providers within the first 3 weeks postpartum. This initial assessment should be followed up with ongoing care, as needed—concluding with a comprehensive postpartum visit no later than 12 weeks after birth. ACOG also recommends that the full assessment reviews physician, social and psychological well-being of the mom. The challenge is that 40% of moms miss their postpartum checkup and 1 in 4 American moms return to work within 2 weeks of giving birth. Access to personalized and equitable care is still not in the reach of all American moms. While the recommendation of ACOG is noteworthy, the ability to deploy this plan within our existing health ecosystem—one that has seen great gaps in maternal health inequities—makes it problematic. Medical care is estimated to account for only 10-20 percent of the modifiable contributors to healthy outcomes for a population. The other 80 to 90 percent are sometimes broadly called the SDoH: health-related behaviors, socioeconomic factors, and environmental factors. The current care delivery model and its connection to health inequities has also been extensively researched. In a recent article by Alex Peahl looking at the impact on black women's experience with prenatal care delivery, they found “care would be tailored to individual patients by using care navigators, flexible models, and colocation of services to reduce barriers”. Hospitals and care providers are beginning to capture social determinants of health data, yet there is an ongoing challenge in connecting social support with improved clinical outcomes. The Deloitte Center for Health Solutions released a report where they surveyed 300 hospitals and health systems to understand how they were addressing and investing in the social needs of their patient populations. They found that “80 percent of hospital respondents reported that leadership is committed to establishing and developing processes to systematically address social needs as part of clinical care . . . however, [their] findings also indicate that much activity is still ad hoc (defined in our survey as occasional and only reaching some of the target population), and gaps remain in connecting initiatives that improve health outcomes or reduce costs”.

Maternal health is a critical aspect of healthcare that encompasses the well-being of women during pregnancy, childbirth, and the postpartum period. Despite advancements in medical care, maternal mortality and morbidity remain significant challenges globally, particularly in underserved communities. Women who experience complications during pregnancy face increased risks of developing long-term health issues, including cardiovascular diseases.

The healthcare industry has recognized that a substantial portion of health disparities are linked to factors beyond direct medical care. These factors, often referred to as social determinants of health, play a considerable role in shaping health outcomes. Traditional healthcare models have struggled to address these non-medical aspects effectively, leading to gaps in care and persistent health inequities.

With the advent of digital technologies, there has been a growing interest in leveraging these tools to enhance healthcare delivery and patient engagement. Digital health platforms have emerged as potential solutions to bridge the gap between clinical care and the everyday lives of patients. These platforms aim to facilitate continuous monitoring, provide educational resources, and enable better communication between patients and healthcare providers.

However, existing digital health solutions often face limitations in their ability to comprehensively address the complex needs of women's health, particularly in the context of maternal care. Many platforms lack integration with medical devices, fail to incorporate social determinants of health, or do not provide adequate support for care coordination among various healthcare professionals and support networks.

Furthermore, the collection and utilization of real-time health data outside of clinical settings remain challenging. There is a need for systems that can effectively capture both clinical and social insights to provide a more holistic view of a patient's health status. Additionally, the ability to stratify this data to identify and address health disparities is an area that requires further development.

As the healthcare landscape continues to evolve towards value-based care models, there is an increasing demand for interoperable solutions that can serve as digital front doors for healthcare entities. These solutions should not only improve patient outcomes but also help reduce overutilization of healthcare resources and bridge access gaps, particularly for women at risk of conditions such as hypertension, diabetes, and mental health issues.

The development of comprehensive digital health engagement platforms tailored to women's health needs represents an opportunity to address these challenges and improve maternal health outcomes on a broader scale.

### BRIEF SUMMARY OF THE INVENTION

The instant invention in one form is directed to a digital health engagement platform for improving the health and care coordination of women suffering from high blood pressure, diabetes and mental health conditions from pre-conception to menopause.

The invention is a digital health engagement platform to help monitor and coordinate support for women at risk for hypertension, behavioral health and diabetes. The tool has a user application and SaaS client portal that captures real time clinical and social insights of patients/health consumers outside of the walls of the clinic/hospital. It serves as an interoperable digital front door solution for healthcare entities, supporting their value-based care strategy to reduce maternal mortality and morbidity. Health consumers can manage their care plans within a digital care village. Health providers and professionals can receive real time alerts and health equity data to drive improvement in outcomes, reduce overutilization and bridge access gaps.

The heart of the system leverages our custom-designed health culture index (HCI) built within the application. The HCI measures health non-medical drivers of health directly from the health consumers' voice via a text enabled engagement model. The HCI measure drives aspects of the care platform including, a risk stratification based on social determinants, an individualized care plan using machine learning, and a clinical insights dashboard for the healthcare team to support proactive interventions. Often, innovative tools like wearable technologies are cost prohibitive to the patient communities who are in the greatest need of the solution. The application according to the present invention in the screenshots of FIGS. 3-12 is designed to serve as a technology bridge between the health consumer and their digital care village. This digital village—including roles for a physician, community health worker, midwife, therapist and loved one—would allow real time connectivity. Those assigned to a care team role would receive alerts and have real time access to clinical and social insights. This level of connectivity creates a proactive pathway of engagement—before the patient reaches an emergent state that leads to over-utilization of resources. In addition, our invention stratifies this patient/consumer data by health equity—offering a more comprehensive and proactive approach to care coordination and health disparities improvement planning.

A digital care village engages a network of health systems, health plans, employers, community-based organizations, pharmaceutical companies, and other entities within the health ecosystem that desire to engage and support the direct health outcomes improvement for women —from pre-conception to menopause. The care village is personalized by the individual member (i.e. health consumer). Bi-directional communication is facilitated through: member care profile alerts, real time health improvement monitoring and referral/resource recommendations.

Members having access to the app can track and monitor the health status of a recently delivered child—showing the connection between mom and baby.

The system could be adapted to work with a variety of Bluetooth enabled medical devices, not just a blood pressure monitor. For example, it could work with a glucose monitor for diabetes management, a heart rate monitor for cardiovascular health, or a wearable fitness tracker for overall wellness monitoring. The specific device could be chosen based on the user's specific health conditions and monitoring requirements. In addition, the system could be adapted to monitor and predict stress.

The digital health engagement platform could include additional functionalities beyond those listed. For example, it could include a symptom tracker, a medication reminder system, a diet and exercise planner, or a mental health support tool. The specific functionalities could be customized based on the user's specific health conditions and care management requirements. Additional functionalities would include care coordination and virtual visits with a clinical team.

The index tool could include different types of question sets beyond the multi-question set described. For example, it could include a single-question set for quick assessments, a visual analog scale for subjective assessments, or a Likert scale for more nuanced assessments. The specific type of question set could be chosen based on the specific non-medical drivers of health being measured.

The system could be integrated with different types of electronic health record systems, not just one. For example, it could be integrated with a hospital's electronic health record system, a primary care physician's electronic health record system, or a specialist's electronic health record system. The specific type of electronic health record system could be chosen based on the user's specific healthcare providers and care coordination requirements.

The system could be adapted to support different types of user interaction methods, not just a client portal. For example, it could support a mobile app, a web-based interface, or a voice-activated assistant. The specific type of user interaction method could be chosen based on the user's specific preferences and accessibility requirements.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a side view of a user interacting with a smartphone and Bluetooth device, according to aspects of the present disclosure.

FIG. 2 depicts a block diagram of a digital health system, according to an embodiment.

FIG. 3 shows a dashboard interface of a digital health engagement platform, according to aspects of the present disclosure.

FIG. 4 illustrates a chat-style health assessment interface of the digital health engagement platform, according to an embodiment.

FIG. 5 depicts a health data monitoring interface displaying blood pressure and kidney health data, according to aspects of the present disclosure.

FIG. 6 shows a digital health village interface displaying care team members, according to an embodiment.

FIG. 7 illustrates a clinical dashboard interface displaying patient information and health metrics, according to aspects of the present disclosure.

FIG. 8 depicts a login/signup screen for the digital health platform, according to an embodiment.

FIG. 9 shows a menu interface screen of the digital health application, according to aspects of the present disclosure.

FIG. 10 depicts a survey invitation screen of the health engagement application, according to aspects of the present disclosure.

FIG. 11 shows a results screen displaying an overall culture score, according to an embodiment.

FIG. 12 illustrates a health culture index dashboard displayed on a laptop computer screen, according to aspects of the present disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms village and circle may be interchangeable in terms such as digital care village or digital care village members and would be interchangeable with digital care circle or digital care circle members, respectively.

The present invention is a digital health engagement platform for monitoring and coordinating the health and social determinant needs of women from pre-conception to menopause. The digital health engagement platform improves the health and care coordination of women suffering from high blood pressure, diabetes and mental health conditions from pre-conception to menopause.

There has never been a more critical time to address the growing maternal health crisis. Women who survive a complicated pregnancy are twice as likely to develop heart disease and other complications. With only 10-20% of health disparities connected to direct medical care, our invention captures clinical and social insights outside of the walls of the clinic. With our digital health platform, women struggling with high blood pressure, mental health issues and/or diabetes can manage their health with a smart assist care plan—connecting directly with their care team in real time.

The present invention is a digital health engagement platform reduces both maternal mortality and maternal morbidity at a global scale. For the 3.7 million births that occurred in 2021, as many as 480,000 women—or more than 1 in 8—experienced maternal morbidity. Research shows us that only 10 to 20 percent of health disparities can be addressed with direct medical care. As such, 80% of disparities must be addressed outside of the walls of hospitals and clinics—often succumbing to a fragmented health ecosystem and sending us into a continuous cycle of poor outcomes. With the rise of the consumer voice and the digital health transformation, an opportunity exists in leverage an engaged health consumer audience as a partner in their own health management while sharing their digital biomarkers with their personal care team to predict and mitigate risk.

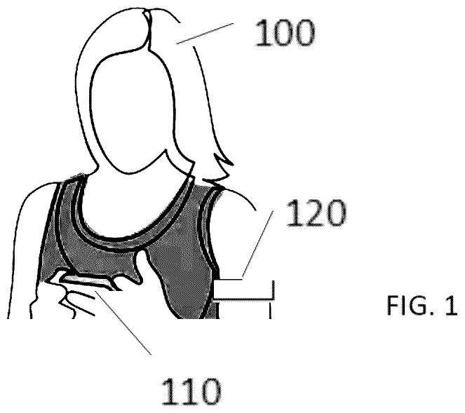

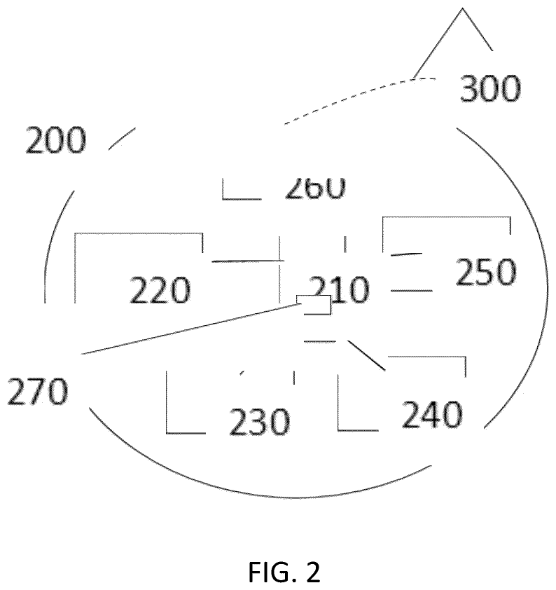

FIG. 1 shows a user 100 accessing a smartphone 110 which includes an app and a Bluetooth device 120 in one embodiment of the digital health engagement platform for monitoring and coordinating the health and social determinant needs of women from pre-conception to menopause. FIG. 2 shows a system 200 for improving the health and care coordination of women suffering from high blood pressure, diabetes and mental health conditions from pre-conception to menopause. The system includes a computer readable medium, which may be a smart phone 210, comprising instructions which, when executed by a processor, cause the computer readable medium to carry out the steps of accessing a digital health engagement platform having at least one module for performing health risk scoring, care plan management, care support, client reporting or interoperability. There may be any number of modules in the platform, some of which include a health risk scoring module 220, care plan management module 230, care support module 240, client reporting module 250 and interoperability module 260. The system includes a Bluetooth enabled medical device such as a blood pressure monitor for tracking and logging specific data that required to monitor overall health of a user wherein the Bluetooth enabled medical device is in communication with the computer readable medium. The system includes an index tool 270 for measuring non-medical drivers of health, the index tool including a multi-question set that is pushed directly from a client portal to the application. The system 200 integrates with an electronic health record system 300 and preferable with the electronic health record system of the United States and globally.

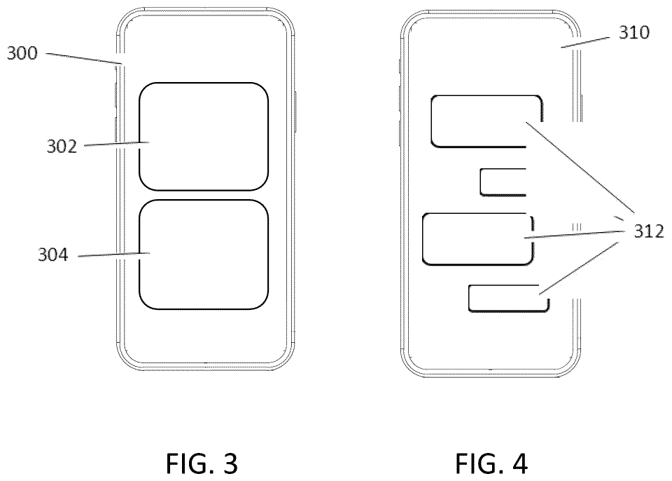

FIG. 3 shows a graphic display 300 on a mobile device including a dashboard interface 304 of the digital health engagement platform. The dashboard interface 304 includes selections to perform specific tasks or to bring the user to a different location within the interface. A to-do list interface 302 is shown above the dashboard interface 304

FIG. 4 illustrates a chat-style health assessment interface 310 of the digital health engagement platform. The health assessment interface 310 may chat with the user by human or automated responses and prompts.

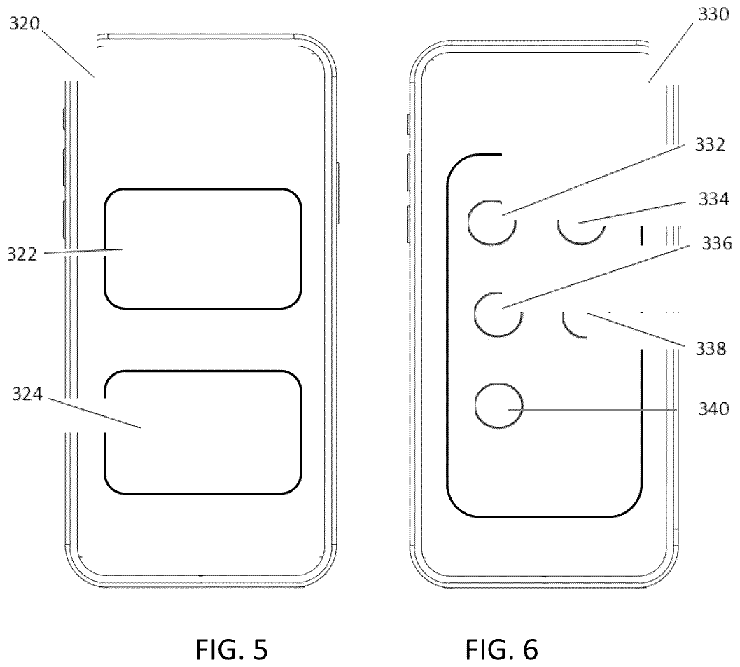

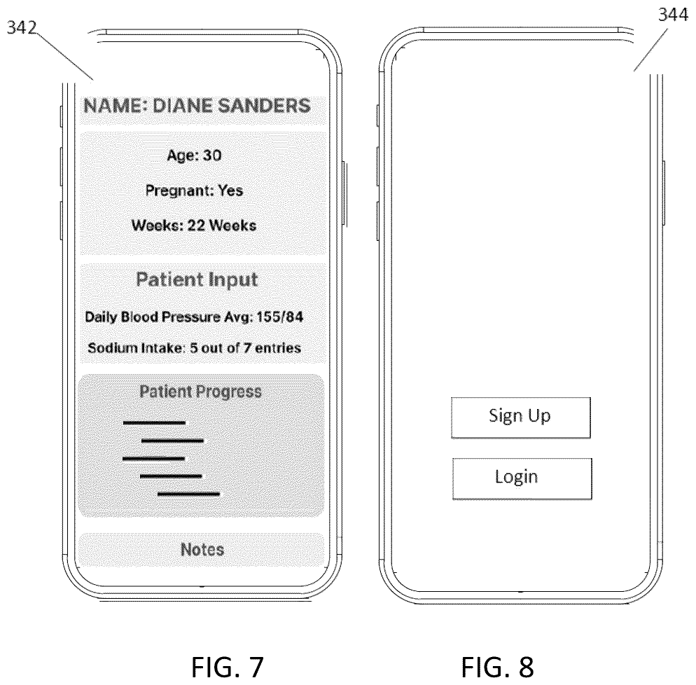

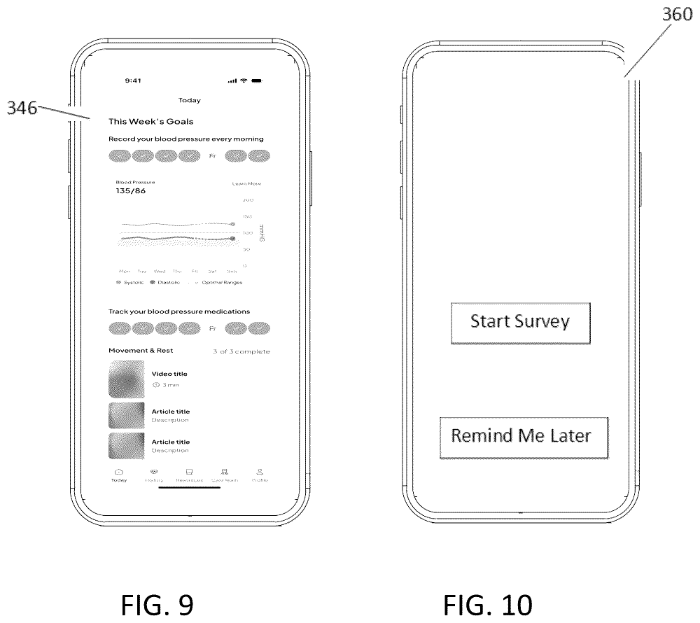

FIG. 5 depicts a health data monitoring interface 320 displaying blood pressure data 322 and kidney health data 324. FIG. 6 shows a digital health village interface 330 displaying a favorites contact list including family physician 332, loved one 334, OB/GYN physician 336, therapist 338, or an add button 340 for adding a favorite contact. FIG. 7 illustrates a clinical dashboard interface 342 displaying patient information and health metrics. FIG. 8 shows a login/signup screen 344 for the digital health platform. FIG. 9 shows a menu interface weekly summary screen 346 which includes interface choices such as daily blood pressure tracking, lifestyle educational resources—along with a menu that includes today's summary, history, resources, care team profile, and logout.

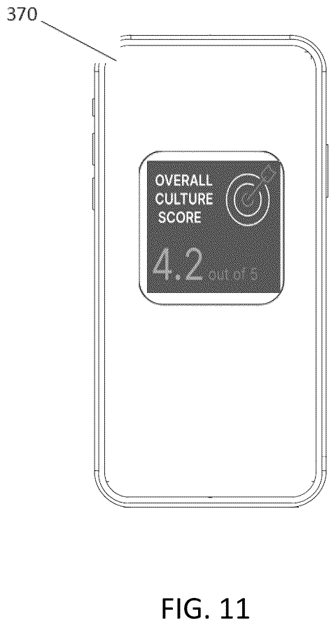

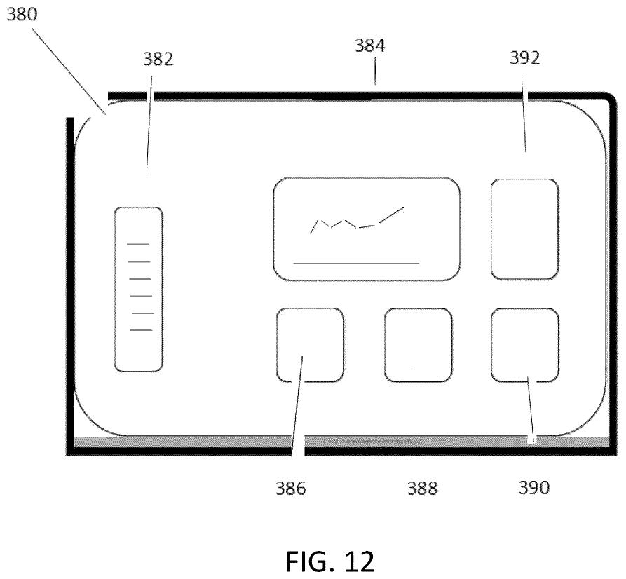

FIG. 10 depicts a survey invitation screen 360 of the health engagement application, according to aspects of the present disclosure. FIG. 11 shows a results screen 370 displaying an overall culture score, according to an embodiment. FIG. 12 illustrates a health culture index dashboard 382 displayed on a laptop computer screen 380. The laptop computer screen 380 includes selection buttons 382 which may direct the user to other modules within the health engagement application including statistics, reports, setting, notifications, messages, help screen or the user to logout of the system. A score chart 384 may be shown by various time intervals along with a short-term improvement indicator 386, total responses 388 and overall culture score 390. Key domains 392 may include health equity, values, risk and access details.

The primary modules perform the tasks of the invention including health risk scoring, care plan management, care support, client reporting and interoperability; and respectively are performed by the health risk scoring module 220, the care plan management module 230, the care support module 240, the client reporting module 250 and the interoperability module 260.

For health risk scoring, the health consumer (member) is invited to download the application directly to their smartphone from Apple AppStore or Google Play store. The first task the member would perform is the completion of a health risk assessment. Upon completing this interactive survey, the member receives a risk score of high, medium or low.

For care plan management, the member is prompted to select at least 1 goal and begin managing their goals and entering various health and social data as requested and based on their selected goal. For ease of use, users will be able to connect a Bluetooth enabled medical device, i.e.: a blood pressure monitor, to automatically track and log specific data that is needed to monitor their overall health.

For care support, the member receives care support through an engaged and personalized digital care village. Through the tool, individuals can connect directly with a physician, therapist, doula and/or loved one. Depending on their role type, the care village member will have a specific level of access.

In considering access:

Care Team has access to all data tracked, demographic data and assessment risk levels.
This should only be available to Provider Organization Clinical Members Community Level is access to their social alerts and basic demographic profile.
   Friend Level is access to demographic data, and care plan/goals

From the task list, members select “Connect to Your Care Circle”, clicks+ADD to add a member and follows the prompts. Members can add a maximum of five (5) members to their circle. For professional members, why would have already been enrolled by their organization and available through a lookup functionality.

Add Care Circle Member:

### Role (Options: Physician/Office, Doula, Midwife, Therapist, Loved One, Community Health Worker, Other)

Name
Email
Organization Look Up
Access Level (Care Team Level, Community Resource Level, Friend Level)

When inviting a loved one to the care circle, the member will add the name and an automated email invitation will be sent for person to download the user application and enroll as a care village member. Their identity will be verified, and they will receive a confirmation email with their unique set up code to complete their set up within the application. Once they are logged in with this code, the member who invited them will be able to confirm that they want them to be a member of their village.

Data and privacy measures are extremely vital with the use of any digital health tool. With this invention, members will be able to reconfirm that they are allowing each member of their digital care village to have access.

Health professional members of the care team (i.e.: physicians, doulas, etc.) will be able to communicate directly with the enrolled member and recommend health literacy resources as well as community referrals to support them on their journey.

In regards to client reporting, organizational clients have access to the SaaS client portal. Within the portal, the client can invite their designated members to download the app, monitor and engage with those members regarding their health goals and alerts, review health equity reports and view anonymized risk trends to better understand how to support their enrolled members.

Within the invention is an index tool that measures non-medical drivers of health. At the core, the tool includes a set of questions that captures the social and structural determinants of health at the community level. The Index data is published on a public facing website—but the community Index scores are mapped to the users of the platform. The community level HCI score captures:
economic indicators of health outcomes at the state and community level—including investment in health promoting resources;
health system access indicators;
physical environment indicators—as it relates to health promoting resources; and
individual health behavior indicators—including utilization, health literacy and prevalence of chronic disease and mortality rates.

Interoperability—The system integrates with electronic health record systems within the United States and globally.

While building the invention, specific coding standards were followed to facilitate plans for interoperability. This means that the enrolled organizations' designated employees who have been connected to a care village will be able to see real time insights directly from that electronic medical record. Notes specific to community referrals, phone calls and other support given can be made directly into that member's medical record.

The present disclosure relates to a digital health engagement platform for improving the health and care coordination of women. In some cases, the platform may focus on women suffering from high blood pressure, diabetes and mental health conditions from pre-conception to menopause. The platform may provide tools for monitoring and coordinating support for women at risk for various health conditions.

In some implementations, the digital health engagement platform may include a user application and a client portal. The platform may capture real-time clinical and social insights of patients outside of traditional healthcare settings. The platform may serve as an interoperable digital solution for healthcare entities to support value-based care strategies aimed at reducing maternal mortality and morbidity.

The platform may allow health consumers to manage their care plans within a digital environment. Healthcare providers and professionals may receive real-time alerts and health equity data through the platform. This data may be used to drive improvements in outcomes, reduce overutilization of resources, and address access gaps in care.

A key component of the system may be a health culture index built within the application. This index may measure non-medical drivers of health directly from health consumers through a text-enabled engagement model. The health culture index may inform various aspects of the care platform, including risk stratification, individualized care planning, and clinical insights for the healthcare team.

The platform may be designed to serve as a technology bridge between health consumers and their care network. This network may include roles for various healthcare professionals and personal contacts. The platform may allow real-time connectivity and information sharing among authorized members of a patient's care team.

In some implementations, the platform may stratify patient/consumer data by health equity factors. This approach may enable a more comprehensive and proactive strategy for care coordination and addressing health disparities.

The digital health engagement platform may be adaptable to work with various Bluetooth-enabled medical devices for health monitoring. The platform may also include additional functionalities beyond basic health tracking, such as symptom monitoring or medication reminders. These features may be customized based on individual user needs and health conditions.

The digital health system 200 may include a smartphone device 210 that serves as a central hub for various health-related functionalities. In some cases, the smartphone device 210 may interact with multiple modules to provide comprehensive health management capabilities.

As shown in FIG. 2, the digital health system 200 may include a health risk module 220, a care plan module 230, a care support module 240, a client reporting module 250, and an interoperability module 260. These modules may be connected to the smartphone device 210 through communication pathways, allowing for seamless data exchange and integration of various health management functions.

The health risk module 220 may be responsible for assessing and analyzing potential health risks for users. In some implementations, the care plan module 230 may generate and manage personalized care plans based on the user's health profile and risk assessment. The care support module 240 may provide ongoing assistance and resources to help users adhere to their care plans.

The client reporting module 250 may generate reports and analytics for healthcare providers or other authorized parties. In some cases, the interoperability module 260 may facilitate data exchange between the digital health system 200 and external systems, such as a health record system 300.

A health index tool 270 may be integrated into the digital health system 200. The health index tool 270 may interact with various modules to collect and analyze health-related data, providing a comprehensive view of the user's health status.

In some implementations, the digital health system 200 may include additional functionalities to enhance user experience and health management. For example, the system may incorporate a symptom tracker to monitor and record user-reported symptoms over time. A medication reminder system may be included to help users adhere to their prescribed medication schedules.

The digital health system 200 may also feature a diet and exercise planner to assist users in maintaining a healthy lifestyle. In some cases, a mental health support tool may be integrated to provide resources and tracking for mental well-being.

The smartphone device 210 may be adapted to work with various Bluetooth-enabled medical devices. While a blood pressure monitor may be one example, the system may also be compatible with other devices. For instance, a glucose monitor may be used for diabetes management, allowing users to track and log blood glucose levels directly through the smartphone device 210.

In some implementations, the digital health system 200 may support integration with a heart rate monitor for cardiovascular health tracking. Additionally, the system may be compatible with wearable fitness trackers, enabling users to monitor overall wellness metrics such as steps taken, calories burned, or sleep patterns.

By incorporating these various modules, tools, and device compatibilities, the digital health system 200 may provide a comprehensive platform for health management and monitoring. The system's modular structure may allow for flexibility and customization based on individual user needs and health conditions.

The digital health system 200 may include a user interface accessible through the smartphone device 210. In some cases, a user FIG. 100 may interact with a smartphone 110 to access various features of the digital health system 200, as illustrated in FIG. 1. The smartphone 110 may display a dashboard interface providing an overview of the user's health information and available tools.

In some implementations, the dashboard interface may include sections for health assessment, care plan management, and access to a digital health village. The health risk module 220 may utilize the health assessment feature to gather information about the user's current health status and potential risk factors. Users may input data directly through the smartphone 110 or, in some cases, data may be automatically collected from a Bluetooth device 120 such as a wearable health monitor.

The care plan module 230 may generate personalized care plans based on the health assessment results. These care plans may be displayed on the smartphone 110, allowing users to track their progress and receive reminders for health-related tasks. In some cases, the digital health system 200 may include functionality for tracking and monitoring the health status of a recently delivered child, providing a connection between maternal and infant health data.

The digital health village feature, managed by the care support module 240, may allow users to connect with healthcare providers, family members, and other support individuals. Users may interact with their care team through messaging, video calls, or by sharing health data directly through the smartphone 110.

In some implementations, the digital health system 200 may support different types of user interaction methods beyond a client portal. For example, the system may offer a mobile app interface optimized for smartphone 110 use, a web-based interface accessible through various devices, or a voice-activated assistant for hands-free interaction. These diverse interaction methods may enhance accessibility and user engagement with the digital health system 200.

The client reporting module 250 may generate reports and visualizations of health data, which users can access through the smartphone 110 interface. These reports may help users and their care teams track progress and identify trends in health metrics over time.

Through the interoperability module 260, the digital health system 200 may integrate with external systems, such as the health record system 300. This integration may allow for seamless data exchange between the user's personal health records and the digital health system 200, enhancing the comprehensiveness of the health information available through the smartphone 110 interface.

The digital health system 200 may include features for health monitoring and data collection. In some cases, the smartphone device 210 may display health metrics collected from various sources, including user input and data from connected devices.

The digital health system 200 may track blood pressure measurements. In some implementations, a systolic pressure value and a diastolic pressure value may be displayed on the smartphone device 210. The systolic pressure value may represent the pressure in the arteries when the heart beats, while the diastolic pressure value may represent the pressure in the arteries when the heart is at rest between beats.

In addition to blood pressure, the digital health system 200 may monitor kidney health. A glomerular filtration rate may be displayed on the smartphone device 210. The glomerular filtration rate may provide an estimate of how well the kidneys are filtering waste from the blood.

The health risk module 220 may utilize these health metrics to assess potential risks and inform care planning. In some cases, the care plan module 230 may generate recommendations based on the collected health data.

Data collection for these health metrics may occur through various methods. In some implementations, the user FIG. 100 may manually input data through the smartphone 110. Alternatively, the Bluetooth device 120 may automatically transmit health data to the smartphone device 210. This automated data collection may enhance the accuracy and frequency of health monitoring.

The health index tool 270 may incorporate different types of question sets to gather comprehensive health information. In some cases, a single-question set may be used for quick assessments. This approach may allow for rapid data collection when time is limited or when only specific information is needed.

In other implementations, the health index tool 270 may employ a visual analog scale for subjective assessments. This type of scale may allow users to indicate their perceived health status or symptoms along a continuous line, providing nuanced data that may be difficult to capture with discrete options.

The health index tool 270 may also utilize a Likert scale for more detailed assessments. This type of scale may present users with a range of response options, typically from strongly disagree to strongly agree, allowing for a more granular understanding of user perceptions and experiences.

The client reporting module 250 may generate visualizations and reports based on the collected health data. These reports may be accessible to healthcare providers through the health record system 300, facilitating informed decision-making and personalized care planning.

Through the interoperability module 260, the digital health system 200 may integrate collected health data with external systems. This integration may allow for a more comprehensive view of the user's health status, combining data from various sources to support holistic health management.

The digital health system may include a health culture index (HCI) feature that provides insights into various factors affecting health outcomes. In some cases, the HCI may incorporate a survey process to gather information from users.

The HCI may include a community level score that captures multiple indicators related to health and well-being. In some implementations, the community level HCI score may incorporate economic indicators that reflect the overall financial health of a community. These economic indicators may provide context for understanding potential barriers to healthcare access and utilization.

The community level HCI score may also include health system access indicators. These indicators may reflect the availability and accessibility of healthcare services within a given community. In some cases, the health system access indicators may consider factors such as the number of healthcare facilities, the availability of specialized care, and the presence of telemedicine options.

Physical environment indicators may be another component of the community level HCI score. These indicators may assess aspects of the built and natural environment that can impact health outcomes. In some implementations, physical environment indicators may include factors such as air and water quality, availability of green spaces, and presence of walkable neighborhoods.

The community level HCI score may also incorporate individual health behavior indicators. These indicators may reflect patterns of health-related behaviors within a community, such as rates of physical activity, dietary habits, and preventive healthcare utilization. In some cases, individual health behavior indicators may also include prevalence of chronic diseases and mortality rates.

The digital health system may present HCI results to users through various interfaces. As shown in FIG. 13, a health culture index dashboard may be displayed on a computer screen. The dashboard may include a line graph showing an overall score trend over time, allowing users to visualize changes in their community's health culture index.

In some implementations, the dashboard may display key metrics related to the HCI. These metrics may include an overall health improvement percentage, total number of responses collected, and an overall culture score. The dashboard may also present key domains with associated scores, providing a more detailed breakdown of the HCI components.

The digital health system may offer reporting capabilities for both individual users and healthcare providers. For individual users, the system may generate personalized reports that contextualize their health data within the broader community health landscape. In some cases, these reports may include recommendations for improving personal health based on community-level indicators.

Healthcare providers may access more comprehensive reporting features through the digital health system. These reports may aggregate anonymized data to provide insights into population health trends, identify areas for targeted interventions, and inform resource allocation decisions. In some implementations, healthcare providers may be able to filter and analyze HCI data based on various demographic and geographic parameters.

The health culture index and associated reporting features may enable a data-driven approach to understanding and addressing health disparities at both individual and community levels. By capturing a wide range of health-related indicators, the HCI may provide a holistic view of factors influencing health outcomes, supporting more targeted and effective healthcare strategies.

The digital health system 200 may integrate with various types of electronic health record systems to enhance interoperability and data exchange across different healthcare entities. In some cases, the interoperability module 260 may facilitate connections with multiple electronic health record systems simultaneously.

The digital health system 200 may integrate with a hospital's electronic health record system. In some implementations, this integration may allow for real-time data synchronization between the digital health system 200 and the hospital's records. The interoperability module 260 may ensure secure transmission of patient data, such as vital signs, medication lists, and treatment plans, between the two systems.

In some cases, the digital health system 200 may also connect with a primary care physician's electronic health record system. This integration may enable the primary care physician to access up-to-date patient information collected through the smartphone device 210 or Bluetooth device 120. The health risk module 220 may utilize data from the primary care physician's records to enhance risk assessments and inform care planning.

The digital health system 200 may further integrate with a specialist's electronic health record system. In some implementations, this integration may allow specialists to view relevant patient data collected through the digital health system 200, such as blood pressure readings or symptom reports. The care support module 240 may use data from the specialist's records to provide more targeted support and recommendations to users.

The interoperability module 260 may employ standardized data formats and protocols to ensure seamless communication between the digital health system 200 and various electronic health record systems. In some cases, the module may use Health Level Seven (HL7) standards or Fast Healthcare Interoperability Resources (FHIR) to facilitate data exchange.

The health index tool 270 may incorporate data from integrated electronic health record systems to provide a more comprehensive view of a user's health status. In some implementations, the tool may combine clinical data from electronic health records with user-reported data and information from connected devices to generate more accurate health assessments.

The client reporting module 250 may generate reports that incorporate data from both the digital health system 200 and integrated electronic health record systems. These comprehensive reports may provide healthcare providers with a holistic view of a patient's health status and care history.

By integrating with various types of electronic health record systems, the digital health system 200 may enhance continuity of care and support more informed decision-making across different healthcare settings. The system's interoperability capabilities may facilitate a more connected and efficient healthcare ecosystem.

The digital health system 200 may provide comprehensive care coordination for women from pre-conception to menopause through the functional interaction of its various components. In some cases, the smartphone device 210 may serve as the central hub for user interaction and data collection.

The health risk module 220 may analyze data collected through the smartphone device 210 and connected Bluetooth device 120 to assess potential health risks. In some implementations, this risk assessment may inform the care plan module 230, which may generate personalized care plans tailored to the user's specific health needs and stage of life.

The care support module 240 may facilitate the creation and management of a personalized digital care village within the digital health system 200. In some cases, this digital care village may include roles for various healthcare professionals and personal contacts. The digital care village may include roles for a physician, community health worker, midwife, therapist, and loved one.

The digital health system 200 may implement different levels of access for care village members based on their role type. In some implementations, members with a Care Team role type may have access to all tracked data, demographic information, and assessment risk levels. This level of access may be reserved for provider organization clinical members.

Members with a Community Level role type may have access to social alerts and basic demographic profiles within the digital health system 200. In some cases, members with a Friend Level role type may have access to demographic data and care plan/goals information.

The client reporting module 250 may generate reports and analytics based on the data collected and processed by the digital health system 200. In some implementations, these reports may be accessible to healthcare providers and authorized care village members, facilitating informed decision-making and collaborative care.

The interoperability module 260 may enable data exchange between the digital health system 200 and external systems, such as the health record system 300. This interoperability may allow for a more comprehensive view of the user's health status by integrating data from various sources.

The health index tool 270 may analyze data from multiple components of the digital health system 200 to provide insights into non-medical drivers of health. In some cases, these insights may inform risk assessments, care planning, and support strategies implemented by other modules within the system.

Within the user application, the digital health system 200 may implement video-based visits (telemedicine) through an integrated video tool like Zoom.

Through the coordinated functioning of these components, the digital health system 200 may provide a comprehensive platform for monitoring, supporting, and coordinating care for women throughout various stages of life. The system's ability to integrate multiple data sources, facilitate communication among care team members, and provide personalized care recommendations may contribute to improved health outcomes and care coordination.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A digital health engagement platform for women, comprising:
a computer readable medium including instructions which, when executed by a processor, cause the computer readable medium to carry out steps of accessing a digital health engagement platform;
at least one module for performing health risk scoring, care plan management, care support, client reporting or interoperability;
a Bluetooth enabled medical device for tracking and logging specific data that is required to monitor overall health of a user, wherein the Bluetooth enabled medical device is in communication with the computer readable medium;
an index tool for measuring non-medical drivers of health, the index tool including a multi-question set that is pushed directly from a client portal to an application;
wherein the platform integrates with an electronic health record system.

2. The digital health engagement platform of claim 1, wherein the platform is configured for improving the health and care coordination of women suffering from high blood pressure, diabetes and mental health conditions (stress and anxiety) from pre-conception to menopause.

3. The digital health engagement platform of claim 1, wherein the Bluetooth enabled medical device includes a blood pressure monitor, continuous glucose monitor and stress monitor.

4. The digital health engagement platform of claim 1 including a health risk scoring module, a care plan management module, a care support module, a client reporting module, and an interoperability module.

5. The digital health engagement platform of claim 4 wherein the care support module facilitates creation and management of a personalized digital care circle.

6. The digital health engagement platform of claim 5 wherein the digital care circle includes roles for a physician, a community health worker, a midwife, and a therapist.

7. The digital health engagement platform of claim 6 wherein the platform implements different levels of access for care circle members based on a care circle member role type.

8. A method for digital health engagement for women, the method comprising:
accessing a digital health engagement platform having at least one module for performing health risk scoring, care plan management, care support, client reporting or interoperability;
tracking and logging specific data that is required to monitor overall health of a user using a Bluetooth enabled medical device in communication with a computer readable medium;
measuring non-medical drivers of health using an index tool including a multi-question set that is pushed directly from a client portal to an application; and
integrating with an electronic health record system.

9. The method of claim 8, wherein the Bluetooth enabled medical device includes a blood pressure monitor.

10. The method of claim 8, further comprising generating a personalized care plan based on the health risk scoring and the measured non-medical drivers of health.

11. The method of claim 10, wherein generating the personalized care plan includes utilizing machine learning algorithms to analyze the tracked data and measured non-medical drivers of health.

12. The method of claim 8, further comprising facilitating creation and management of a personalized digital care circle within the digital health engagement platform.

13. The method of claim 12, wherein the digital care circle includes roles for a physician, a community health worker, a midwife, and a therapist.

14. The method of claim 13 including implementing different levels of access for care circle members based on their role type.

15. A system for digital women's health engagement, comprising:
a smartphone device configured to access a digital health engagement platform;
a health risk module for assessing potential health risks;
a care plan module for generating personalized care plans;
a care support module for facilitating creation and management of a digital care circle;
a client reporting module for generating reports and analytics;
an interoperability module for enabling data exchange with external systems; and
a health index tool for analyzing non-medical drivers of health.

16. The system of claim 15, wherein the health risk module is configured to analyze data collected through the smartphone device and a connected Bluetooth enabled medical device to assess potential health risks.

17. The system of claim 15, wherein the care support module is configured to implement different levels of access for care circle members based on their role type.

18. The system of claim 17, wherein the role types include Care Team, Community Level, and Friend Level.

19. The system of claim 18, wherein members with a Care Team role type have access to all tracked data, demographic information, and assessment risk levels.

20. The system of claim 15 wherein the system implements video-based visits (telemedicine) through an integrated video tool within the user application.
